(19) United States
(12) Patent Application Publication
(10) Pub. No.: US 2022/0023253 A1
(43) Pub. Date: Jan. 27, 2022

(54) Cannabinoid Compositions and Methods for Treating Joint Pain and Inflammation

(71) Applicant: NP Pharma Holdings, LLC, Sheridan, WY (US)

(72) Inventor: Peyton Palaio, Denver, CO (US)

(21) Appl. No.: 17/381,441

(22) Filed: Jul. 21, 2021

Related U.S. Application Data
(60) Provisional application No. 63/054,575, filed on Jul. 21, 2020.

(51) Int. Cl. A61K 31/352; A61K 31/05; A61K 38/01; A61K 35/57

(52) U.S. Cl. CPC A61K 31/352; A61K 35/57; A61K 38/014; A61K 31/05

(57) ABSTRACT

Provided are compositions comprising a cannabinoid and collagen, and methods of making and using such compositions. The disclosed compositions can be useful in, for example, treating joint pain and inflammation (e.g., joint pain associated with arthritis).

### CROSS-REFERENCE TO RELATED APPLICATIONS

This application claims priority to U.S. Provisional Application No. 63/054,575, filed Jul. 21, 2020, which is incorporated by reference in its entirety.

### BACKGROUND

Joint pain is a common symptom originating from a variety of underlying conditions, including osteoarthritis, rheumatoid arthritis, bursitis, gout, and injuries such as strains and sprains. Joint pain can affect any part of a subject and can be widespread or generalized, as is often the case with fibromyalgia and chronic fatigue syndrome. Even in subjects with otherwise healthy joints, overuse and exercise-induced joint pain is common, particularly as the subject ages. In the United States, for instance, studies have shown that nearly a quarter of the population is affected by joint pain, often attributed to an underlying condition such as arthritis.

Despite the prevalence of joint pain, contributing pathologies are poorly understood, making effective treatment a challenge. Clinical osteoarthritis, for example, was once thought to be primarily a disease of the cartilage, but recent research has shown that the condition involves multiple tissue pathologies. Thus, there remains a need for effective treatment of joint pain and inflammation. These needs and others are met by the following compositions and methods.

### SUMMARY

In one aspect, this disclosure relates to cannabinoid compositions and methods of making and using the compositions for treating joint pain and inflammation, including pain originating from an underlying condition such as arthritis, as well as pain originating from injuries and overuse, such as exercise-induced joint pain.

Disclosed are compositions comprising a cannabinoid in an amount of from about 1 wt % to about 20 wt % based on the total weight of the composition; and a collagen.

Also disclosed are compositions comprising a cannabinoid, a collagen, and eggshell membrane.

Also disclosed are compositions comprising a cannabinoid and a collagen, wherein the composition is substantially free of turmeric, ginger, Burseraceae, and/or skullcap.

Also disclosed are compositions comprising a cannabinoid and a collagen in an amount of from about 11 wt % to about 20 wt % based on the total weight of the composition.

Also disclosed are compositions comprising a cannabinoid in an amount of from about 1 wt % to about 20 wt % based on the total weight of the composition; undenatured type II collagen in an amount of from about 2 wt % to about 20 wt % based on the total weight of the composition; and eggshell membrane in an amount of from about 1 wt % to about 20 wt % based on the total weight of the composition; wherein the composition is substantially free of turmeric, ginger, Burseraceae, and/or skullcap.

Also disclosed are methods for making a disclosed composition.

Also disclosed are methods for treating joint pain or inflammation in a subject, e.g., pain or inflammation originating from a condition such as arthritis, comprising administering an effective amount of a disclosed composition to the subject.

Also disclosed are kits comprising a disclosed composition and one or more of an agent known for treating arthritis; and instructions for treating arthritis.

Still other objects and advantages of the present disclosure will become readily apparent by those skilled in the art from the following detailed description, which is shown and described by reference to preferred aspects, simply by way of illustration of the best mode. As will be realized, the disclosure is capable of other and different aspects, and its several details are capable of modifications in various respects, without departing from the disclosure. Accordingly, the description is to be regarded as illustrative in nature and not as restrictive.

### DETAILED DESCRIPTION

The present invention can be understood more readily by reference to the following detailed description of the invention and the Examples included therein.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present compositions, methods, and kits may be understood more readily by reference to the following detailed description of preferred embodiments and the examples included therein.

While aspects of this disclosure can be described and claimed in a particular statutory class, such as the system statutory class, this is for convenience only and one of skill in the art will understand that each aspect of this disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present application is not entitled to antedate such publication by virtue of prior invention. Further, stated publication dates may be different from actual publication dates, which can require independent confirmation.

### A. DEFINITIONS

Listed below are definitions of various terms used to describe this invention. These definitions apply to the terms as they are used throughout this specification, unless otherwise limited in specific instances, either individually or as part of a larger group.

As used in the specification and in the claims, the term “comprising” can include the aspects “consisting of” and “consisting essentially of.”

As used in the specification and claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where said event or circumstance occurs and instances where it does not.

As used herein, the term “by weight,” when used in conjunction with a component, unless specially stated to the contrary is based on the total weight of the formulation or composition in which the component is included. For example, if a particular element or component in a composition or article is said to have 8% by weight, it is understood that this percentage is in relation to a total compositional percentage of 100%.

A weight percent of a component, or weight %, or wt %, unless specifically stated to the contrary, is based on the total weight of the formulation or composition in which the component is included.

As used herein, the term “subject” can be a vertebrate, such as a mammal, a fish, a bird, a reptile, or an amphibian. Thus, the subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with an ailment, disease, or disorder. The term “patient” includes human and veterinary subjects.

As used herein, the term “treatment” refers to the medical management of a patient with the intent to cure, ameliorate, stabilize, or prevent an ailment, disease, pathological condition, disorder, or injury. This term includes active treatment, that is, treatment directed specifically toward the improvement of a disease, pathological condition, disorder, or injury, and also includes causal treatment, that is, treatment directed toward removal of the cause of the associated disease, pathological condition, disorder, or injury. In addition, this term includes palliative treatment, that is, treatment designed for the relief of symptoms rather than the curing of the disease, pathological condition, disorder, or injury; preventative treatment, that is, treatment directed to minimizing or partially or completely inhibiting the development of the associated disease, pathological condition, disorder, or injury; and supportive treatment, that is, treatment employed to supplement another specific therapy directed toward the improvement of the associated disease, pathological condition, disorder, or injury. In various aspects, the term covers any treatment of a subject, including a mammal (e.g., a human), and includes: (i) preventing the disorder or condition from occurring in a subject that can be predisposed to the disorder or condition but has not yet been diagnosed as having it; (ii) inhibiting the disorder or condition, i.e., arresting its development or exacerbation thereof; or (iii) relieving the disorder or condition, i.e., promoting healing of the disorder or condition. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compositions or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed composition or a product of a disclosed method of making, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed composition or a product of a disclosed method of making, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-HIV agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

References in the specification and concluding claims to parts by weight of a particular element or component in a composition or article, denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

As used herein, the term “substantially,” in, for example, the context “substantially free of” refers to a composition having less than about 10% by weight, e.g., less than about 5%, less than about 1%, less than about 0.5%, less than about 0.1%, less than about 0.05%, or less than about 0.01% by weight of the stated material, based on the total weight of the composition.

It is further understood that the term “substantially,” when used in reference to a composition, refers to at least about 60% by weight, e.g., at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components can be present in any ratio predetermined by one of ordinary skill in the art.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

Disclosed are also components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

### B. COMPOSITIONS

In various aspects, the disclosed compositions comprise (a) a cannabinoid, (b) a collagen, and other optional ingredients including, for example, eggshell membrane, and a pharmaceutically-acceptable carrier.

### 1. Cannabinoid

Cannabis is a genus of flowering plants that includes at least three species, Cannabis sativa, Cannabis indica, and Cannabis ruderalis. Cannabis plants produce a family of terpeno-phenolic compounds called cannabinoids. More than 100 cannabinoids have been identified from crude cannabis. Most cannabinoids exist in two forms, as acids and in neutral (decarboxylated) forms. The acid form is designated by an “A” at the end of its acronym, e.g., TCHA. Cannabinoids are synthesized in the plant as acid forms, and while some decarboxylation does occur in the plant, it increases significantly post-harvest, and the kinetics of decarboxylation increase at high temperatures. Decarboxylation can be achieved by thorough drying of the plant material followed by heating it or exposing it to light or alkaline conditions.

Without wishing to be bound by any theory, the cellular effects of cannabinoids are thought to be mediated by two G-protein coupled receptors, cannabinoid receptor type 1 (CB1) and CB2. CB1 is present in the central nervous system and other tissues such as the adrenal gland, adipose tissue, heart, liver, lung, prostate, uterus, ovary, testis, bone marrow, thymus, and tonsils. The CB2 receptor is widely expressed in the immune system and can also be found in bone and the gastrointestinal system, among other tissues. In addition to CB receptors, other receptors mediate cellular effects of cannabinoids.

CB and other receptors are expressed in different joint tissues, including neurons, chondrocytes, synovium, and bone. As a result, endocannabinoid signaling likely plays a role in modulating joint pain and inflammation, including pain and inflammation originating from a joint condition such as arthritis. CB1 and CB2 receptors, for instance, have been found in synovial biopsies taken from human subjects undergoing knee arthroplasty for osteoarthritis and rheumatoid arthritis. Other endocannabinoid receptors have been detected in the joints of subjects suffering from arthritis at much higher levels than that seen in control subjects.

Thus, in various aspects, the disclosed compositions include one or more cannabinoids. The cannabinoids can be in the acid or neutral form and can be derived from a cannabis plant or produced synthetically. Thus, in various aspects, the cannabinoid can be a cannabinoid acid. In some aspects, the cannabinoid in the composition can be Δ⁹-tetrahydrocannabinol (Δ⁹-THC), Δ⁸-tetrahydrocannabinol (Δ⁸-THC), cannabichromene (CBC), cannabicyclol (CBL), cannabidiol (CBD), cannabielsoin (CBE), cannabigerol (CBG), cannabinidiol (CBND), cannabinol (CBN), cannabitriol (CBT), or a combination thereof. The cannabinoid in the composition can also be in acid form, e.g., Δ⁹-THCA, Δ⁸-THCA, CBCA, CBLA, CBDA, CBEA, CBGA, CBNDA, CBNA, CBTA, or a combination thereof In some aspects, the cannabinoid is CBD, CBDA, THC, THCA, or a combination thereof In further aspects, the cannabinoid is CBD, THC, or a combination thereof. In further aspects, the cannabinoid is CBD. In still further aspects, the cannabinoid is THC.

In other aspects, the composition is free or substantially free of THC. In some aspects, the composition has less than about 10 wt % THC, less than about 5 wt % THC, less than about 3 wt % THC, less than about 1 wt % THC, less than about 0.8 wt % THC, less than about 0.7 wt % THC, less than about 0.6 wt % THC, less than about 0.5 wt % THC, less than about 0.4 wt % THC, less than about 0.3 wt % THC, less than about 0.2 wt % THC, less than about 0.1 wt % THC, less than about 0.05 wt % THC, or less than about 0.01 wt % THC, based on the total weight of the composition.

In some aspects, the composition is free or substantially free of CBN. In some aspects, the composition has less than about 10 wt % CBN, less than about 5 wt % CBN, less than about 3 wt % CBN, less than about 1 wt % CBN, less than about 0.8 wt % CBN, less than about 0.7 wt % CBN, less than about 0.6 wt % CBN, less than about 0.5 wt % CBN, less than about 0.4 wt % CBN, less than about 0.3 wt % CBN, less than about 0.2 wt % CBN, less than about 0.1 wt % CBN, less than about 0.05 wt % CBN, or less than about 0.01 wt % CBN, based on the total weight of the composition.

In further aspects, the composition is free or substantially free of THC and CBN. Thus, in various further aspects, the composition has less than about 10 wt %, less than about 5 wt %, less than about 3 wt %, less than about 1 wt %, less than about 0.8 wt %, less than about 0.7 wt %, less than about 0.6 wt %, less than about 0.5 wt %, less than about 0.4 wt %, less than about 0.3 wt %, less than about 0.2 wt %, less than about 0.1 wt %, less than about 0.05 wt %, or less than about 0.01 wt %, of both THC and CBN, based on the total weight of the composition.

In various aspects, the cannabinoid is present in the composition in an amount of from about 1 wt % to about 20 wt % based on the total weight of the composition. In some aspects, the cannabinoid is present in the composition in an amount of from about 2 wt % to about 20 wt % based on the total weight of the composition. In some aspects, the cannabinoid is present in the composition in an amount of from about 3 wt % to about 20 wt % based on the total weight of the composition. In some aspects, the cannabinoid is present in the composition in an amount of from about 5 wt % to about 20 wt % based on the total weight of the composition. In some aspects, the cannabinoid is present in the composition in an amount of from about 8 wt % to about 20 wt % based on the total weight of the composition. In some aspects, the cannabinoid is present in the composition in an amount of from about 10 wt % to about 20 wt % based on the total weight of the composition. In some aspects, the cannabinoid is present in the composition in an amount of from about 12 wt % to about 20 wt % based on the total weight of the composition. In some aspects, the cannabinoid is present in the composition in an amount of from about 15 wt % to about 20 wt % based on the total weight of the composition. In some aspects, the cannabinoid is present in the composition in an amount of from about 18 wt % to about 20 wt % based on the total weight of the composition.

In some aspects, the composition can include about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt % of the cannabinoid, based on the total weight of the composition.

In various aspects, the one or more cannabinoids in the composition can be obtained commercially, prepared synthetically, or extracted from a cannabis plant. Once harvested, cannabis plant material typically includes flowers, leaves, and/or stems. In some aspects, cannabis plant material can be frozen for a suitable period of time, e.g., 36 hours, prior to being dried and extracted. Once dried, cannabis plant material can be extracted using a variety of techniques, including hydrocarbon extraction and supercritical CO₂ extraction.

In some aspects, the compositions comprise neutral cannabinoids, which can be prepared by decarboxylating cannabinoid acids. In various aspects, cannabinoid acids obtained from cannabis plant material can be decarboxylated by heating the dried plant material at a temperature of about 220° F. for at least 10-15 minutes followed by heating for about 280° F. for at least 45 minutes. Other known methods for decarboxylating cannabinoid acids from cannabis plant material can also be used.

### 2. Collagen

In various aspects, the disclosed compositions also include a collagen. Without wishing to be bound by any theory, collagen is believed to affect immunological tolerance to antigens involved in joint conditions such as arthritis and may also reduce T-cell attack on healthy cartilage. In general, collagen is the principal protein component of the extra-cellular matrix. In mammals, collagen can constitute as much as 60% of the total body protein. Collagen is found in the skin, tendons, bones and teeth, and occurs as fibrous inclusions in most other structures of a subject. Animal collagen types I through XIX have been discovered. Of these collagen types, types I through V are most commonly used in medical and cosmetic applications and in food supplements.

In various aspects, the collagen in the compositions can be type I through type V collagen. Type I collagen is the major fibrillar collagen of bone and skin, comprising approximately 80-90% of an animal's total collagen. Type I collagen is the major structural macromolecule present in the extracellular matrix of multicellular organisms and comprises approximately 20% of total protein mass. Type I collagen is a heterotrimeric molecule comprising two alpha 1(I) chains and one alpha 2(I) chain. Type II collagen is a homotrimeric collagen comprising three identical alpha 1(II) chains. Type III collagen is a major fibrillar collagen found in skin and vascular tissues. Type III collagen is a homotrimeric collagen comprising three identical alpha 1(III) chains. Type IV collagen is found in basement membranes in the form of sheets rather than fibrils. Most commonly, type IV collagen contains two alpha 1 (IV) chains and one alpha 2(IV) chain. Type V collagen is a fibrillar collagen found in, primarily, bones, tendon, cornea, skin, and blood vessels. Type V collagen exists in both homotrimeric and heterotrimeric forms. One form of type V collagen is a heterotrimer of two alpha 1 (V) chains and one alpha 2(V) chain. Another form of type V collagen is a heterotrimer of alpha 1(V), alpha 2(V), and alpha 3(V) chains. A further form of type V collagen is a homotrimer of alpha 1(V). In various aspects, the collagen can be an undenatured collagen.

In various aspects, collagen can be obtained from an animal source. Some native collagen animal sources include, but are not limited to, avians or birds, ungulates or hoofed mammals, such as for example, horses, cows, goats, pigs, sheep, deer, non-ungulates or mammals without hoofs; marsupials, such as opossums, kangaroos, wombats, and bandicoots; marine animals or any animal that lives in water, such as whales, dolphins, swordfish, tuna, shark, mahimahi, sailfish, marlin, yellowtail, escolar, lancet fish, mackerel, salmon, cod, flounder, bass, or sturgeon; amphibians, such as for example, frogs, toads, salamanders; reptiles, such as snakes, crocodiles, alligators, or combinations thereof. In some aspects, the collagen is obtained from a marine animal.

In some aspects, the collagen can be obtained from any body part of the animal that has collagen. For example, collagen may be obtained from skin, tendons, ligaments, or bone. In some aspects, the collagen source is from a marine animal and the body part is from the tendon, such as caudal, caudal ray, pectoral, and/or inter-costal tendon.

In some aspects, the collagen is type II collagen. In further aspects, the collagen is undenatured type II collagen. In various aspects, undenatured type II collagen can be derived from a variety of mammalian sources such as avian sources. In further aspects, poultry cartilage such as chicken cartilage can be a source of undenatured type II collagen. In other aspects, undenatured type II collagen can be obtained from other warm-blooded animal tissue containing Type II collagen, such as turkey or bovine cartilage.

In various aspects, the collagen is present in the composition in an amount of from about 2 wt % to about 20 wt % based on the total weight of the composition. In some aspects, the collagen is present in the composition in an amount of from about 3 wt % to about 20 wt % based on the total weight of the composition. In some aspects, the collagen is present in the composition in an amount of from about 5 wt % to about 20 wt % based on the total weight of the composition. In some aspects, the collagen is present in the composition in an amount of from about 8 wt % to about 20 wt % based on the total weight of the composition. In some aspects, the collagen is present in the composition in an amount of from about 11 wt % to about 20 wt % based on the total weight of the composition. In some aspects, the collagen is present in the composition in an amount of from about 15 wt % to about 20 wt % based on the total weight of the composition. In some aspects, the collagen is present in the composition in an amount of from about 18 wt % to about 20 wt % based on the total weight of the composition.

In some aspects, the composition can include about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt % of collagen, based on the total weight of the composition.

In various aspects, undenatured collagen is present in the composition in an amount of from about 2 wt % to about 20 wt % based on the total weight of the composition. In some aspects, the undenatured collagen is present in the composition in an amount of from about 3 wt % to about 20 wt % based on the total weight of the composition. In some aspects, the undenatured collagen is present in the composition in an amount of from about 5 wt % to about 20 wt % based on the total weight of the composition. In some aspects, the undenatured collagen is present in the composition in an amount of from about 8 wt % to about 20 wt % based on the total weight of the composition. In some aspects, the undenatured collagen is present in the composition in an amount of from about 11 wt % to about 20 wt % based on the total weight of the composition. In some aspects, the undenatured collagen is present in the composition in an amount of from about 15 wt % to about 20 wt % based on the total weight of the composition. In some aspects, the undenatured collagen is present in the composition in an amount of from about 18 wt % to about 20 wt % based on the total weight of the composition.

In some aspects, the composition can include about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt % of undenatured collagen, based on the total weight of the composition.

In various aspects, undenatured type II collagen is present in the composition in an amount of from about 2 wt % to about 20 wt % based on the total weight of the composition. In some aspects, the undenatured type II collagen is present in the composition in an amount of from about 3 wt % to about 20 wt % based on the total weight of the composition. In some aspects, the undenatured type II collagen is present in the composition in an amount of from about 5 wt % to about 20 wt % based on the total weight of the composition. In some aspects, the undenatured type II collagen is present in the composition in an amount of from about 8 wt % to about 20 wt % based on the total weight of the composition. In some aspects, the undenatured type II collagen is present in the composition in an amount of from about 11 wt % to about 20 wt % based on the total weight of the composition. In some aspects, the undenatured type II collagen is present in the composition in an amount of from about 15 wt % to about 20 wt % based on the total weight of the composition. In some aspects, the undenatured type II collagen is present in the composition in an amount of from about 18 wt % to about 20 wt % based on the total weight of the composition.

In some aspects, the composition can include about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt % of undenatured type II collagen, based on the total weight of the composition.

In further aspects, the composition consists essentially of the cannabinoid and the collagen. In some aspects, the composition consists essentially of the cannabinoid, the collagen, and a pharmaceutically-acceptable carrier. In other aspects, the composition consists essentially of the cannabinoid and undenatured collagen. In further aspects, the composition consists essentially of the cannabinoid, undenatured collagen, and a pharmaceutically-acceptable carrier. In still further aspects, the composition consists essentially of the cannabinoid and undenatured type II collagen. In some aspects, the composition consists essentially of the cannabinoid, undenatured type II collagen, and a pharmaceutically-acceptable carrier.

### 3. Eggshell Membrane

Eggshell membrane contains gycosaminoglycans and proteins, among other components, useful for maintaining healthy articular cartilage and the surrounding synovium. Thus, in various aspects, the compositions comprise eggshell membrane or a material derived from eggshell membrane. In general, eggshell membrane is comprised of two individual membranes between egg albumin and eggshell. These membranes include protein fibers. The proteins in eggshell membrane include, without limitation, arginine, glutamic acid, methionine, histidine, cystine, and proline. Eggshell membrane can also include hydroxyproline, hydroxylysine, desmosine, and various collagens. Other components of eggshell membrane include acid glycosaminoglycans, including dermatan sulfate and chondroitin sulfate, sulfated glycoproteins, hyaluronic acid, and ovotransferrin.

In some aspects, the eggshell membrane in the compositions can be processed eggshell membrane, eggshell membrane isolates, eggshell membrane hydroysates, eggshell membrane powder, or combinations thereof In some aspects, the eggshell membrane can be in liquid, semi-solid, or solid form, such as, for example, partially deydrated powder form comprising varying amounts of liquid or moisture.

In various aspects, the eggshell membrane is present in the composition in an amount of from about 1 wt % to about 20 wt % based on the total weight of the composition. In some aspects, the eggshell membrane is present in the composition in an amount of from about 2 wt % to about 20 wt % based on the total weight of the composition. In some aspects, the eggshell membrane is present in the composition in an amount of from about 3 wt % to about 20 wt % based on the total weight of the composition. In some aspects, the eggshell membrane is present in the composition in an amount of from about 5 wt % to about 20 wt % based on the total weight of the composition. In some aspects, the eggshell membrane is present in the composition in an amount of from about 8 wt % to about 20 wt % based on the total weight of the composition. In some aspects, the eggshell membrane is present in the composition in an amount of from about 10 wt % to about 20 wt % based on the total weight of the composition. In some aspects, the eggshell membrane is present in the composition in an amount of from about 12 wt % to about 20 wt % based on the total weight of the composition. In some aspects, the eggshell membrane is present in the composition in an amount of from about 15 wt % to about 20 wt % based on the total weight of the composition. In some aspects, the eggshell membrane is present in the composition in an amount of from about 18 wt % to about 20 wt % based on the total weight of the composition.

In some aspects, the composition can include about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt % eggshell membrane, based on the total weight of the composition.

In further aspects, the composition consists essentially of the cannabinoid, the collagen, and the eggshell membrane. In some aspects, the composition consists essentially of the cannabinoid, the collagen, eggshell membrane, and a pharmaceutically-acceptable carrier. In other aspects, the composition consists essentially of the cannabinoid, undenatured collagen, and eggshell membrane. In further aspects, the composition consists essentially of the cannabinoid, undenatured collagen, eggshell membrane, and a pharmaceutically-acceptable carrier. In still further aspects, the composition consists essentially of the cannabinoid, undenatured type II collagen, and eggshell membrane. In some aspects, the composition consists essentially of the cannabinoid, undenatured type II collagen, eggshell membrane, and a pharmaceutically-acceptable carrier.

In various aspects, the eggshell membrane can be obtained by methods known in the art, including methods that include the step of separation the eggshell membrane from the egg yolk, egg white, and eggshell prior to subsequent processing and isolation steps. In some aspects, the source of eggshell membrane can be from cracked eggs, where the eggshell membrane is still attached to the eggshell. In further aspects, the eggshell membrane can be separated from the eggshell by any convenient method. In various aspects, methods for separating eggshell membrane from eggshell can include a mechanical separation, for instance, by rolling and pulling membranes away from washed shells after removal of the yoke and albumen of fresh or uncooked eggs. In further aspects, eggshell membrane can be separated from eggshell by agitating coarsely chopped eggshells containing membranes in the presence of an acid until the membrane separates from the shell.

In some aspects, the separation method can also include the step of deydrating the separated eggshell membrane to produce eggshell membrane flakes of various sizes and dimensions. In further aspects, the method for isolating eggshell membrane can include the step of powdering eggshell membrane flakes to produce an eggshell membrane with a suitable particle size. Powdering can be accomplished using standard milling or pulverizing procedures.

In further aspects, powdered eggshell membrane can be subjected to enzymatic hydrolysis using a yeast enzyme. The slurry resulting from this method can include a soluble fraction containing, among other things, hyaluronic acid and other water-soluble fractions and an insoluble fraction comprising collagen and other components.

### 4. Pharmaceutically-Acceptable Carrier

In various aspects, the compositions comprise a pharmaceutically-acceptable carrier. In some aspects, the composition is a topical composition, formulated for oral use, or as a cream, ointment, or gel. Thus, in some aspects, the pharmaceutically-acceptable carrier can be a carrier suitable for the manufacture of a topical composition, oral form, cream, ointment, or gel.

In some aspects, the pharmaceutically-acceptable carrier can include an excipient. Suitable excipients include, without limitation, saccharides, for example, glucose, lactose, or sucrose, mannitol, or sorbitol, cellulose derivatives, and/or calcium phosphate, for example, tricalcium phosphate or acidic calcium phosphate.

In further aspects, the pharmaceutically-acceptable carrier can include a binder. Suitable binders include, without limitation, tare compounds such as starch paste, for example, corn, wheat, rice, and potato starch, gelatin, tragacanth, methylcellulose, hydroxypropyl methylcellulose, carboxymethylcellulose, and/or polyvinylpyrrolidone. In still further aspects, there can be a disintegrating agent, such as the aforementioned starches and carboxymethyl starch, crosslinked polyvinylpyrrolidone, agar, or alginic acid or a salt thereof, such as sodium alginate.

In some aspects, the pharmaceutically-acceptable carrier can include an additive. Examples of additives include, but are not limited to, diluents, buffers, binders, surface-active agents, lubricants, humectants, pH adjusting agents, preservatives (including anti-oxidants), emulsifiers, occlusive agents, opacifiers, antioxidants, colorants, flavoring agents, gelling agents, thickening agents, stabilizers, and surfactants, among others. Thus, in various further aspects, the additive is vitamin E, gum acacia, citric acid, stevia extract powder, Luo Han Gou, Monoammonium Glycyrhizinate, Ammonium Glycyrrhizinate, honey, or combinations thereof. In a still further aspect, the additive is a flavoring agent, a binder, a disintegrant, a bulking agent, or silica. In a further aspect, the additive can include flowability-control agents and lubricants, such as silicon dioxide, talc, stearic acid and salts thereof, such as magnesium stearate or calcium stearate, and/or propylene glycol.

In various aspects, when the composition is formulated for oral use, such as for example, a tablet, pill, or capsule, the composition can include a coating layer that is resistant to gastric acid. Such a layer, in various aspects, can include a concentrated solution of saccharides that can comprise gum arabic, talc, polyvinylpyrrolidone, polyethylene glycol, and/or titanium dioxide, and suitable organic solvents or salts thereof.

In some aspects, when the composition is formulated as a topical composition such as a cream or ointment, the pharmaceutically-acceptable carrier can comprise a cream or ointment base, such as hydrocarbon ointment bases, white Vaseline, yellow Vaseline (Vaselinum album and Vaselinum flavum), Vaseline oil (Oleum Vaselini), white ointment, and liquid ointment (Unguentum album and Unguentum flavum). In further aspects, a cream or ointment can include solid paraffin or wax, an additive providing a firmer texture, absorptive ointment bases, hydrophilic Vaseline (Vaselinum hydrophylicum), lanoline (Lanolinum), and cold cream (Unguentum leniens). In some aspects, a cream or ointment can include water-removable ointment bases, such as hydrophilic ointment (Unguentum hydrophylum), water-soluble ointment bases, such as polyethylene glycol ointment (Unguentum Glycolis Polyaethyleni), bentonite bases, and other suitable components. In some aspects, a cream or ointment can include methylcellulose, carboxymethylcellulose sodium salt, oxypropylcellulose, polyethylene glycol, polyethylene oxide, or carbopol.

### 5. Compositions Free of Turmeric, Ginger, Burseraceae, and/or Skullcap

In some aspects, the composition is free or substantially free of turmeric, ginger, Burseraceae, and/or skullcap, including compositions that are free or substantially free of any component of turmeric, ginger, Burseraceae, and/or skullcap. In some aspects, the composition has less than about 10 wt % turmeric, ginger, Burseraceae, and/or skullcap, less than about 5 wt % turmeric, ginger, Burseraceae, and/or skullcap, less than about 3 wt % turmeric, ginger, Burseraceae, and/or skullcap, less than about 1 wt % turmeric, ginger, Burseraceae, and/or skullcap, less than about 0.8 wt % turmeric, ginger, Burseraceae, and/or skullcap, less than about 0.7 wt % turmeric, ginger, Burseraceae, and/or skullcap, less than about 0.6 wt % turmeric, ginger, Burseraceae, and/or skullcap, less than about 0.5 wt % turmeric, ginger, Burseraceae, and/or skullcap, less than about 0.4 wt % turmeric, ginger, Burseraceae, and/or skullcap, less than about 0.3 wt % turmeric, ginger, Burseraceae, and/or skullcap, less than about 0.2 wt % turmeric, ginger, Burseraceae, and/or skullcap, less than about 0.1 wt % turmeric, ginger, Burseraceae, and/or skullcap, less than about 0.05 wt % turmeric, ginger, Burseraceae, and/or skullcap, or less than about 0.01 wt % turmeric, ginger, Burseraceae, and/or skullcap, based on the total weight of the composition.

In some aspects, the composition is free or substantially free of turmeric or any component thereof. In some aspects, the composition has less than about 10 wt % turmeric, less than about 5 wt % turmeric, less than about 3 wt % turmeric, less than about 1 wt % turmeric, less than about 0.8 wt % turmeric, less than about 0.7 wt % turmeric, less than about 0.6 wt % turmeric, less than about 0.5 wt % turmeric, less than about 0.4 wt % turmeric, less than about 0.3 wt % turmeric, less than about 0.2 wt % turmeric, less than about 0.1 wt % turmeric, less than about 0.05 wt % turmeric, or less than about 0.01 wt % turmeric, based on the total weight of the composition.

In some aspects, the composition is free or substantially free of ginger or any component thereof. In some aspects, the composition has less than about 10 wt % ginger, less than about 5 wt % ginger, less than about 3 wt % ginger, less than about 1 wt % ginger, less than about 0.8 wt % ginger, less than about 0.7 wt % ginger, less than about 0.6 wt % ginger, less than about 0.5 wt % ginger, less than about 0.4 wt % ginger, less than about 0.3 wt % ginger, less than about 0.2 wt % ginger, less than about 0.1 wt % ginger, less than about 0.05 wt % ginger, or less than about 0.01 wt % ginger, based on the total weight of the composition.

In some aspects, the composition is free or substantially free of Burseraceae or any component thereof. In some aspects, the composition has less than about 10 wt % Burseraceae, less than about 5 wt % Burseraceae, less than about 3 wt % Burseraceae, less than about 1 wt % Burseraceae, less than about 0.8 wt % Burseraceae, less than about 0.7 wt % Burseraceae, less than about 0.6 wt % Burseraceae, less than about 0.5 wt % Burseraceae, less than about 0.4 wt % Burseraceae, less than about 0.3 wt % Burseraceae, less than about 0.2 wt % Burseraceae, less than about 0.1 wt % Burseraceae, less than about 0.05 wt % Burseraceae, or less than about 0.01 wt % Burseraceae, based on the total weight of the composition.

In some aspects, the composition is free or substantially free of skullcap or any component thereof. In some aspects, the composition has less than about 10 wt % skullcap, less than about 5 wt % skullcap, less than about 3 wt % skullcap, less than about 1 wt % skullcap, less than about 0.8 wt % skullcap, less than about 0.7 wt % skullcap, less than about 0.6 wt % skullcap, less than about 0.5 wt % skullcap, less than about 0.4 wt % skullcap, less than about 0.3 wt % skullcap, less than about 0.2 wt % skullcap, less than about 0.1 wt % skullcap, less than about 0.05 wt % skullcap, or less than about 0.01 wt % skullcap, based on the total weight of the composition.

### C. METHODS OF MAKING THE COMPOSITIONS

In one aspect, disclosed are methods of making a disclosed composition. Thus, in various aspects, disclosed are methods for making a composition, the method comprising the step of combining: (a) the cannabinoid, (b) the collagen, and (c) other optional components including, for example, the eggshell membrane or pharmaceutically-acceptable carrier. The compositions can be prepared using methods known in the art.

In some aspects, the method of making comprises combining an amount of cannabinoid, collagen, and other optional components including, for example, the eggshell membrane or pharmaceutically-acceptable carrier, such that the amount of cannabinoid in the composition is from about 1 wt % to about 20 wt %, based on the total weight of the composition. In a further aspect, the method of making comprises combining an amount of cannabinoid, collagen, and other optional components including, for example, the eggshell membrane or pharmaceutically-acceptable carrier, such that the amount of collagen in the composition is from about 2 wt % to about 20 wt %, based on the total weight of the composition. In a further aspect, the method of making comprises combining an amount of cannabinoid, collagen, and other optional components including, for example, the eggshell membrane or pharmaceutically-acceptable carrier, such that the amount of collagen in the composition is from about 11 wt % to about 20 wt %, based on the total weight of the composition.

In a further aspect, the method of making comprises combining an amount of cannabinoid, collagen, eggshell membrane, and other optional components including, for example, the pharmaceutically-acceptable carrier, such that (a) the amount of cannabinoid in the composition is from about 1 wt % to about 20 wt %, based on the total weight of the composition, (b) the amount of collagen in the composition is from about 2 wt % to about 20 wt %, based on the total weight of the composition, and (c) the amount of eggshell membrane in the composition is from about 1 wt % to about 20 wt %, based on the total weight of the composition.

It is understood that the disclosed compositions can be employed in the disclosed methods of using.

### D. METHODS OF USING THE COMPOSITIONS

### 1. Treatment Methods

The disclosed compositions are useful in treating joint pain and inflammation, including pain originating from an underlying condition such as arthritis, as well as pain originating from injuries and overuse, such as exercise-induced joint pain. In some aspects, the treatment methods are useful in treating arthritis, including, for example, rheumatoid arthritis and osteoarthritis.

The compositions can be administered to the subject via, for example, oral administration (e.g., as a tablet, capsule, lozenge, or troche) or topical administration (e.g., as a cream, ointment, or gel). Alternatively, the compositions can be administered via a device such as, for example, a transdermal patch. Administration can be continuous or intermittent. A preparation can be administered therapeutically; that is, administered to treat or ameliorate an existing disorder or condition.

The effective amount or dosage of the composition or an ingredient thereof can vary within wide limits. Such a dosage is adjusted to the individual requirements in each particular case including the specific composition(s) being administered and the condition being treated, as well as the patient being treated. In general, single dose compositions can contain such amounts or submultiples thereof of the composition to make up the daily dose. The dosage can be adjusted by the individual physician in the event of any contraindications. Dosage can vary, and can be administered in one or more dose administrations daily, for one or several days. In some aspects, the effective amount is a therapeutically-effective amount. In a further aspect, the effective amount is a prophylactically-effective amount.

In a further aspect, the methods comprise administering to a subject an effective amount of a disclosed composition. In some aspects, the subject is a mammal. In a further aspect, the subject is a human. In a still further aspect, the subject has been diagnosed with a need for treatment of joint pain and inflammation, including pain originating from an underlying condition such as arthritis, as well as pain originating from injuries and overuse, such as exercise-induced joint pain, prior to the administering step. In a still further aspect, the subject has been diagnosed with a need for treatment of arthritis prior to the administering step.

In a further aspect, the treatment method comprises the step of identifying a subject in need of treatment of joint pain and inflammation, including pain originating from an underlying condition such as arthritis, as well as pain originating from injuries and overuse, such as exercise-induced joint pain.

In a still further aspect, the treatment method comprises administering at least one agent known for the treatment of joint pain and inflammation, including pain originating from an underlying condition such as arthritis, as well as pain originating from injuries and overuse. In some aspects, for example, the treatment method comprises administering at least one agent known for the treatment of arthritis, such as, for example, a nonsteroidal anti-inflammatory drug (NSAID), a disease modifying antirheumatic drug (DMARD), a biologic response modifier, a corticosteroid, or a combination thereof. Suitable NSAIDs include, for example, aspirin, ibuprofen, naproxen, ketoprofen, oxaprozin, indomethacin, etodolac, nabumetone, naproxen/esomeprazole, magnesium, diclofenac, or a combination thereof. Suitable DMARDs include, for example, hydroxychloroquine, sulfasalazine, leflunomide, methotrexate, minocycline, azathioprine, cyclosporine, tofacitinib, or a combination thereof. Suitable biologic response modifiers include, for example, abatecept, adalimumab, anakinra, certolizumab, etanercept, etanercept-szzzs, golimumab, infliximab, rituximab, tocilizumab, or a combination thereof. Suitable corticosteroids include, for example, cortisone, decadron, delta-cortef, deltasone, dexamethasone, hydrocortone, kenacort, Medrol, methylprednisolone, orasone, prednisolone, prednisone, triamcinolone, aristocort, celestone, cinalone, depo-medrol, hydeltrasol, hydeltra TBA, kenalog, or a combination thereof.

In various aspects, the step of administering the at least one agent known for the treatment of joint pain and inflammation, including pain originating from an underlying condition such as arthritis, as well as pain originating from injuries and overuse, can be simultaneous with the administration of a disclosed composition. In other aspects, the agent and the disclosed composition are administered sequentially. In a further aspect, the agent and the disclosed composition are co-formulated. In another aspect, the agent and the disclosed composition are not co-formulated.

### 2. Other Uses

In one aspect, the disclosure relates to the use of a disclosed composition or a product of a disclosed method. In a further aspect, a use relates to the manufacture of a medicament for the treatment of joint pain and inflammation, including pain originating from an underlying condition such as arthritis, as well as pain originating from injuries and overuse, such as exercise-induced joint pain. In some aspects, a use relates to the manufacture of a medicament for the treatment of arthritis, including, for example, rheumatoid arthritis and osteoarthritis.

In a further aspect, the use relates to a process for preparing a pharmaceutical composition comprising an effective amount of a disclosed composition or a product of a disclosed method of making, for use as a medicament. In some aspects, an effective amount is a therapeutically-effective amount.

In a further aspect, the use relates to a process for preparing a pharmaceutical composition comprising a therapeutically effective amount of a disclosed composition or a product of a disclosed method of making, wherein a pharmaceutically acceptable carrier is intimately mixed with a therapeutically effective amount of the composition or the product of a disclosed method of making.

In various aspects, the use relates to a treatment of joint pain and inflammation, including pain originating from an underlying condition such as arthritis, as well as pain originating from injuries and overuse, such as exercise-induced joint pain. In some aspects, a use relates to the manufacture of a medicament for the treatment of arthritis, including, for example, rheumatoid arthritis and osteoarthritis. In one aspect, the use is characterized in that the subject is a human.

It is understood that the disclosed uses can be employed in connection with the disclosed compositions, products of disclosed methods of making, methods, and kits. In a further aspect, the disclosure relates to the use of a disclosed composition or a disclosed product in the manufacture of a medicament for treating joint pain and inflammation, including pain originating from an underlying condition such as arthritis, as well as pain originating from injuries and overuse, such as exercise-induced joint pain, in a mammal. In a further aspect, the disclosure relates to the use of a disclosed composition or a disclosed product in the manufacture of a medicament for treating arthritis, including, for example, rheumatoid arthritis and osteoarthritis, in a mammal.

### 3. Manufacture of a Medicament

In one aspect, the disclosure relates to a method for the manufacture of a medicament for treating joint pain and inflammation, including pain originating from an underlying condition such as arthritis, as well as pain originating from injuries and overuse, such as exercise-induced joint pain, the method comprising combining a therapeutically effective amount of a disclosed composition or product of a disclosed method with a pharmaceutically acceptable carrier or diluent. In a further aspect, the disclosure relates to a method for the manufacture of a medicament for treating arthritis, including, for example, rheumatoid arthritis and osteoarthritis, the method comprising combining a therapeutically effective amount of a disclosed composition or product of a disclosed method with a pharmaceutically acceptable carrier or diluent.

The present method includes the administration to an animal, particularly a mammal, and more particularly a human, of a therapeutically effective amount of the composition effective in the treatment of the disorder. The dose administered to an animal, particularly a human, in the context of the present disclosure should be sufficient to affect a therapeutic response in the animal over a reasonable time frame. One skilled in the art will recognize that dosage will depend upon a variety of factors including the condition of the animal and the body weight of the animal.

The size of the dose also will be determined by the route, timing, and frequency of administration as well as the existence, nature, and extent of any adverse side effects that might accompany the administration of the composition and the desired physiological effect. It will be appreciated by one of skill in the art that various conditions or disorders, in particular chronic conditions or disorders, may require prolonged treatment involving multiple administrations.

### E. KITS

In one aspect, disclosed are kits comprising a disclosed composition. Thus, in various aspects, disclosed are kits comprising a disclosed composition and one or more of (a) an agent known for treating joint pain and inflammation, including pain originating from an underlying condition such as arthritis, as well as pain originating from injuries and overuse, such as exercise-induced joint pain; and (b) instructions for treating the condition or disorder. In a further aspect, disclosed are kits comprising a disclosed composition and one or more of (a) an agent known for treating arthritis; and (b) instructions for treating arthritis.

In one aspect, the kit comprises at least one agent known for the treatment of joint pain and inflammation, including pain originating from an underlying condition such as arthritis, as well as pain originating from injuries and overuse. In some aspects, for example, the treatment method comprises administering at least one agent known for the treatment of arthritis, such as, for example, a nonsteroidal anti-inflammatory drug (NSAID), a disease modifying antirheumatic drug (DMARD), a biologic response modifier, a corticosteroid, or a combination thereof. Suitable NSAIDs include, for example, aspirin, ibuprofen, naproxen, ketoprofen, oxaprozin, indomethacin, etodolac, nabumetone, naproxen/esomeprazole, magnesium, diclofenac, or a combination thereof. Suitable DMARDs include, for example, hydroxychloroquine, sulfasalazine, leflunomide, methotrexate, minocycline, azathioprine, cyclosporine, tofacitinib, or a combination thereof. Suitable biologic response modifiers include, for example, abatecept, adalimumab, anakinra, certolizumab, etanercept, etanercept-szzzs, golimumab, infliximab, rituximab, tocilizumab, or a combination thereof. Suitable corticosteroids include, for example, cortisone, decadron, delta-cortef, deltasone, dexamethasone, hydrocortone, kenacort, Medrol, methylprednisolone, orasone, prednisolone, prednisone, triamcinolone, aristocort, celestone, cinalone, depo-medrol, hydeltrasol, hydeltra TBA, kenalog, or a combination thereof.

In a further aspect, the composition and the agent are co-packaged. In a still further aspect, the composition and the agent are not co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compositions. It is also understood that the disclosed kits can be employed in connection with the disclosed methods of using the compositions.

The previous description of the disclosed aspects is provided to enable any person skilled in the art to make or use the present disclosure. Various modifications to these aspects will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A composition comprising:
(a) a cannabinoid in an amount of from about 1 wt % to about 20 wt % based on the total weight of the composition; and
(b) a collagen.

2. The composition of claim 1, wherein the collagen is present in an amount of from about 2 wt % to about 20 wt % based on the total weight of the composition.

3. The composition of claim 1, wherein the collagen is present in an amount of from about 11 wt % to about 20 wt % based on the total weight of the composition.

4. The composition of claim 1, wherein the composition consists essentially of the cannabinoid and the collagen.

5. The composition of claim 1, wherein the cannabinoid is Δ⁹-tetrahydrocannabinol (Δ⁹-THC), cannabidiol (CBD), cannabinol (CBN), cannabicyclol (CBL), cannabigerol (CBG), cannabichromene (CBC), or a mixture thereof.

6. The composition of claim 1, wherein the cannabinoid is CBD, Δ⁹-THC, or a mixture thereof.

7. The composition of claim 1, wherein the cannabinoid is CBD.

8. The composition of claim 1, wherein the cannabinoid is a cannabinoid acid.

9. The composition of claim 1, wherein the collagen is an undenatured collagen.

10. The composition of claim 1, wherein the collagen is undenatured type II collagen.

11. The composition of claim 1, wherein the composition further comprises eggshell membrane.

12. The composition of claim 11, wherein eggshell membrane is present in an amount of from about 1 wt % to about 20 wt % based on the total weight of the composition.

13. The composition of claim 1, wherein the composition is substantially free of turmeric, ginger, Burseraceae, and/or skullcap.

14. The composition of claim 1, wherein the composition is a topical composition.

15. The composition of claim 1, wherein the composition is formulated for oral use.

16. The composition of claim 1, wherein the composition is formulated as a cream or an ointment.

17. The composition of claim 1, further comprising a pharmaceutically-acceptable carrier.

18. A composition comprising:
(a) a cannabinoid;
(b) a collagen; and
(c) eggshell membrane.

19. The composition of claim 18, wherein the collagen is present in an amount of from about 2 wt % to about 20 wt % based on the total weight of the composition.

20. The composition of claim 18, wherein the composition consists essentially of the cannabinoid, the collagen, and eggshell membrane.
